(19) United States
(12) Patent Application Publication
(10) Pub. No.: US 2010/0093945 A1
(43) Pub. Date: Apr. 15, 2010

(54) DIFFUSION RETARDATION IN FLUOROPLASTICS

(76) Inventor: Karin Jacobson, Nacka (SE)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/526,698

(22) PCT Filed: Feb. 25, 2008
(86) PCT No.: PCT/SE08/50208

§ 371 Date: Nov. 2, 2009

(30) Foreign Application Priority Data
Feb. 23, 2007 (SE) 0700448-4

(51) Int. Cl. C08L 27/16; C08L 27/04; C07D 403/14; C07C 69/76; C07C 39/12

(52) U.S. Cl. 525/374; 525/55; 525/375; 525/384; 544/209; 560/103; 568/744

(57) ABSTRACT

A process of slowing down diffusion of an element or a compound through a fluoroplastic comprising the addition of a reactive additive having reactive groups that react with the element or compound. A process of preventing degradation of the fluoroplastics PVDF and ECTFE used in, or in connection with a reactor where chlorine dioxide is produced comprising mixing the fluoroplastic with a reactive additive having reactive groups. A diffusion resistant fluoroplastic which comprises a reactive additive having reactive groups that react with an element and/or compound to which the fluoroplastic is diffusion resistant. The use of a reactive additive having reactive groups as an additive in a fluoroplastic to prevent or slow down the diffusion of an element or a compound through the fluoroplastic. The use of a reactive additive having reactive groups as an additive in the fluoroplastics PVDF and ECTFE used in, or in connection with a reactor for producing chlorine dioxide to prevent or slow down degradation of the fluoroplastic.

The present invention is concerned with preventing diffusion through fluoropolymers, such as those used as liners to protect metal or FRP structures. It is further concerned with stabilizing fluoropolymers used in or at a chlorine dioxide reactor against deterioration.

### TECHNICAL FIELD

One of the most important differences in properties between plastics and metals, when used in corrosive environments, is the fact that polymers are permeable to small molecules. This permeability can lead to swelling of the polymer and also means that polymers cannot be used as absolute shields. Fluoroplastics are widely used as protective linings and in other shielding applications since they have unique chemical and temperature resistance in severe environments. However, experience from the field shows that the lifetime of a lined or coated structure quite often is determined by diffusion of aggressive species, such as hydrochloric acid (HCl) and chlorine dioxide (ClO₂), through the fluoroplastic material followed by corrosion attack on the FRP (fibreglass reinforced plastic) or steel substrate.

Thus, the fluoroplastics are normally not themselves attacked chemically but small molecules, such as acids and ClO₂, can diffuse through the material and attack the other, less corrosion resistant, material. A decrease of the diffusion rate or the amount of the permeating media would increase the service life of structures where diffusion is a problem, i.a. lined tanks or pipes.

Another problem encountered with one of the fluoroplastics, PVDF, and sometimes also with ECTFE, and not generally recognized occurs when used in, or in connection with a chlorine dioxide reactor. In spite of the general high resistance of fluoroplastics to chlorine and chlorine compounds the fluoroplastic is degraded and must be changed after a certain time. Thus, in this case the fluoroplastic itself is attacked.

In such a reactor many different elements and compounds are present. It is not known which species or combination of species cause the degradation of the fluoroplastic.

### BACKGROUND ART

U.S. Pat. No. 3,557,050 describes a vinyl fluoride polymer which is heat stabilized by a combination of an alkali metal formate and an organic antioxidant.

U.S. Pat. No. 3,557,051 describes heat stabilization of a polymer comprising a homopolymer of vinyl fluoride and a copolymer of vinyl fluoride and up to 25 wt % of copolymerizable monomers by the use of a combination of an inorganic non-metallic reducing agent and an organic antioxidant.

U.S. Pat. No. 3,775,496 describes a pigmented coating composition containing a liquid medium, pigment and a polyvinyl fluoride polymer. The composition is heat stabilized by incorporation of a mixture of an aliphatic polyol, organic antioxidant and glycidyl methacrylate polymer. It is especially underlined that lack of any one of the three components materially mitigates the effect to be otherwise achieved.

However, none of these documents discuss the problem with diffusion through a polymer, nor the problem of degradation in a chlorine dioxide reactor.

### OBJECTS OF THE INVENTION

One object of the invention is to overcome the problems discussed above by preventing or slowing down diffusion through a fluoroplastic, such as a liner on a substrate of steel or FRP.

A further object is to avoid or decrease the degradation of fluoroplastics used in or in connection with a chlorine dioxide reactor.

Another object is to achieve a fluoroplastic which is diffusion resistant or at least shows a decreased diffusion.

Still another object is to achieve a fluoroplastic which is completely or at least partially resistant to degradation when used in or in connection with a chlorine dioxide reactor.

A further object is to achieve a process for producing a fluoroplastic which is diffusion resistant or at least shows a decreased diffusion.

### SHORT DESCRIPTION OF THE INVENTION

One idea for slowing down the diffusion that has come up is the addition of a reactive additive, i.e., an additive which will react with the permeating media without impairing the properties of the polymer.

The objects of the invention are achieved by the addition to the fluoroplastic of an additive having reactive groups which will react with the permeating media without impairing the properties of the fluoropolymer.

It is not necessary to add any further stabilizer, in addition to the additive having reactive groups. Thus, the diffusion resistant fluoropolymers of the invention do not contain for example inorganic non-metallic reducing agents, aliphatic polyols together with glycidyl methacrylate polymer or alkali metal formate in combination with the additive having reactive groups.

Hindered phenols are presently used in thermal stabilizer systems for other plastics. However, fluoroplastics have generally such high strength and thermal resistance that the use of stabilizers has not been necessary. Also, in the present case it is not a question of stabilizing the fluoroplastic, but of hindering diffusion through the polymer.

Hindered phenols are e.g. used in water conduits, normally made of polyolefins, such as polyethylene or polypropylene. The plastic is stabilized from oxidization by oxygen. However, the drinking water contains small amounts of chlorine dioxide or hypochlorite and it has been noticed that the plastic is gradually depleted of the stabilizer owing to its reaction with the chlorine compounds.

Due to this Irganox 1010, one of the most commonly used hindered phenols, was chosen for a trial investigation. PVDF was chosen as the main polymer matrix since this material is often used in liner applications and since the diffusion rate can easily be determined in this material. Some experiments were also performed on ECTFE containing 1 wt % Irganox 1010.

It was found that the addition of Irganox 1010 slowed down the diffusion of chlorine dioxide and HClO by reacting with these compounds. The reaction between the additive and the permeating media could be followed by FTIR. Additive loadings between 0.1 and 1 wt % were tested and it was found to be a linear relationship between amount of additive and penetration depth. The addition of 1 wt % Irganox 1010 to PVDF (polyvinylidene fluoride) gave about half of the penetration depth of ClO₂ as compared to a sample without additive. For HClO the effect was even larger.

In addition to Irganox 1010, another hindered phenol Irganox 1330 has been tested. It was found to give the same good effect on the diffusion rate as Irganox 1010. To check the use of other reactive groups, apart from phenols, Chimasorb 944, which is a hindered amine, was also tested. It also showed the same effect as the hindered phenols.

### DETAILED DESCRIPTION OF THE INVENTION

The invention concerns a process of slowing down diffusion of an element or a compound through a fluoroplastic comprising the addition of a reactive additive that reacts with the element or compound. The element or compound which should be prevented from diffusing through the fluoropolymer may be for instance chlorine or a chlorine compound such as chlorine dioxide or hypochloric acid.

The reactive additive is preferably a hindered phenol or hindered amine. The necessary concentration may be as low as 0.1 wt %, preferably more than 0.5 wt % and especially at least 1 wt %. At most 10 wt % should be used, preferably at most 5 wt %. The best results are obtained at a loading of about 1 wt %.

In this application wt % is based on the weight of the plastic without the additive.

Other possible reactive additives are vitamin E, lignin, and phenols in general.

It was also discovered that not only does the reactive additive slow down diffusion of aggressive species, but it also increases the resistance of PVDF when used in such an aggressive surrounding as in or in connection with a chlorine dioxide reactor.

Suitable reactive additives are hindered phenols and amines such as

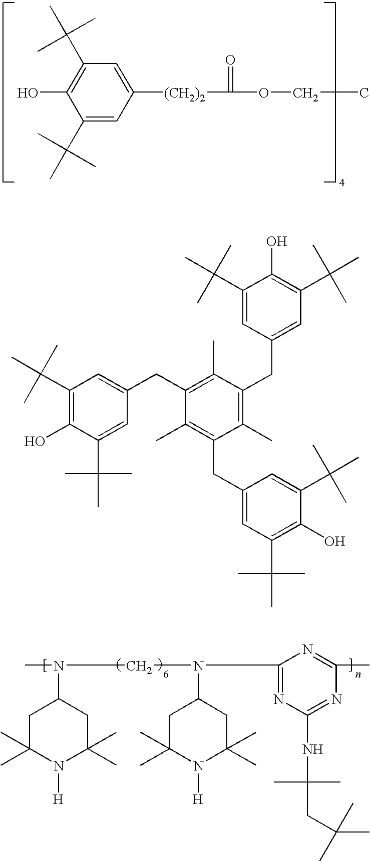

The invention is further clarified by the following description with reference to the enclosed drawings.

### BRIEF DESCRIPTION OF DRAWINGS

FIG. 1 is a diagram showing penetration depth of chlorine dioxide in different plastics.

FIG. 2 is a diagram showing solubility of chlorine dioxide in different thermoplastics.

FIG. 3 is a diagram showing penetration depth of chlorine dioxide, with and without additive.

FIG. 4 is a diagram showing penetration profiles of chlorine dioxide.

FIG. 5 is a diagram showing concentration of penetrating agent for different concentrations of Irganox 1010.

FIG. 6 is a diagram showing Arrhenius plots of the temperature dependence.

FIG. 7 is a diagram showing the penetration depth at different concentrations of chlorine dioxide.

### EXAMPLES

After 5 years in service an FRP pipe with a lining of FEP (fluoroplastic), which has been used for transporting Cl₂ containing brine, could no longer be used since the lining had loosened completely and obstructed the flow. The reason for the loosening was that Cl₂ had diffused through the FEP and destroyed the interface between the FRP and the lining.

Another example of failure due to diffusion through a fluoroplastic liner concerns a PVDF lined FRP pipe for transport of hot chlorine dioxide bleached pulp. This pipe burst open dumping 600 tonnes of hot pulp on the factory floor. The failure was due to stress corrosion cracking occurring as a result of bad clamping generating stresses in the pipe and ClO₂ diffusing through the PVDF attacking the fibre glass in the FRP.

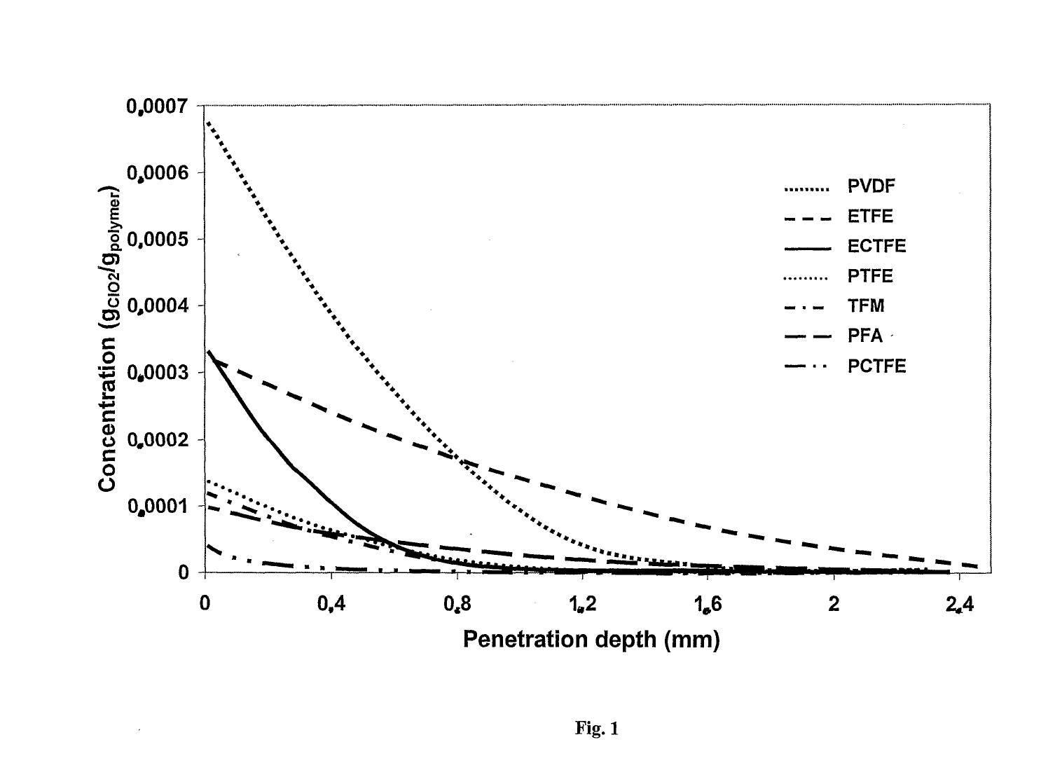

With the help of new techniques developed by the inventor, data on diffusion, permeation and solubility could be generated for different fluoroplastics. It was found that the diffusion is very fast through many of the fluoroplastics, especially PVDF which is one of the most used lining materials. FIG. 1 shows the concentration of ClO₂ as a function of penetration depth, measured after immersion of the plastic at 70° C. for 24 hours.

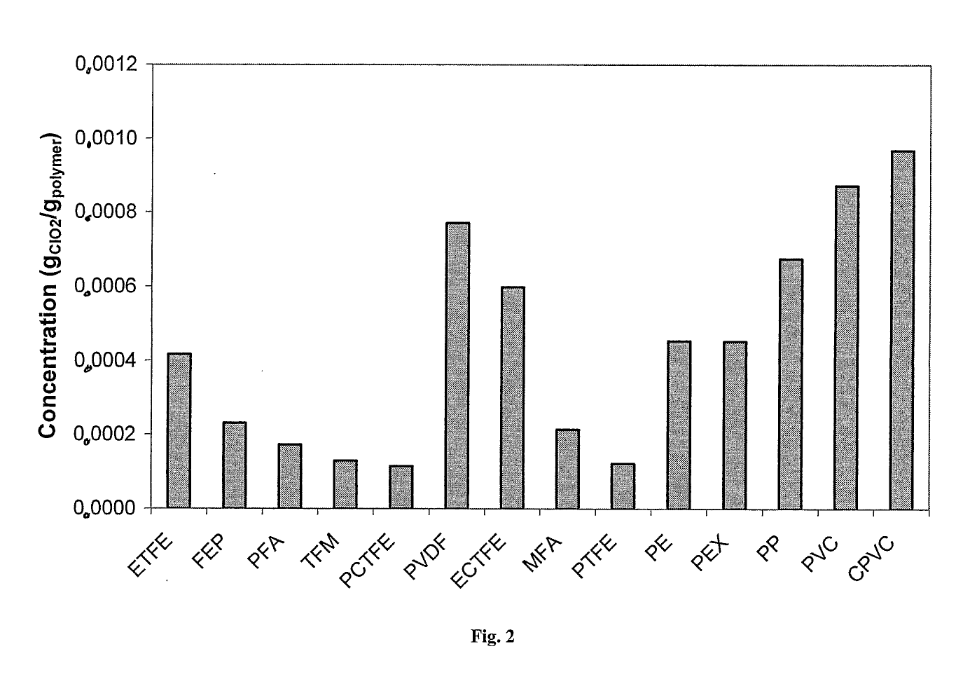

Of importance is, however, not only how fast but also how much that is permeating. This will depend on the solubility. FIG. 2 shows the solubility of ClO₂ in different thermoplastics, at different temperatures. As can be seen, the solubility is quite large in PVDF compared to the all the others, except for PVC and CPVC.

It is well known that a chemical reaction will slow down the permeation through a plastic material. Since fluoroplastics are so chemically inert in themselves the penetrant will pass through without being held back by chemical reactions. In other plastics it might be that the penetrant is diffusing very slowly due to it reacting with the polymer or some additive. The material will thus act as a shield for a while but it might become totally degraded and useless with time. One way of getting the reduced diffusion rate without impairing the properties of the polymer is by adding a reactive additive, i.e., an additive which can react with the permeating media without changing the properties of the polymer.

One additive that could be suitable as a reactive additive for preventing ClO₂ diffusion is Irganox 1010. This is a hindered phenol commonly used as an antioxidant for polyolefins (PP and PE). It is easily available at a relatively low price. Another additive that could be of interest is lignin. The reaction between lignin and ClO₂ is well known from paper bleaching. It is important that the additive is stable at the processing temperature of the polymer, which is not the case for the combination of Irganox 1010 with the fully fluorinated polymers such as PFA. In this case an additive with a higher decomposition temperature should be used. An example of such an additive is Irganox E201 (Vitamine E).

The aim of a laboratory study was to investigate if the addition of a reactive additive could slow down the diffusion of chlorine dioxide in PVDF.

A commercial grade of PVDF powder (SOLEF 1010) was supplied by Solvay Solexis and Irganox 1010, Formula I, was supplied by Ciba Specialty Chemicals. The Irganox was mixed into the PVDF powder by first dissolving different amounts in dichloromethane (CH₂Cl₂) and then adding it to the PVDF powder. The mixtures were stirred thoroughly and left to dry for 48 h.

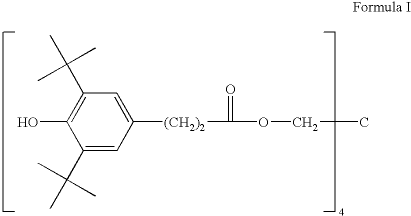

Sheets with different concentrations of Irganox 1010 were then prepared by compression moulding of the powder.

In addition to the above mentioned samples, four batches of plaques (100×100×3 mm) were injection moulded from granulated PVDF (SOLEF 1010). Batch 1 had an addition of 1 wt % of Irganox 1010, batch 2 1 wt % Irganox 1330, which is also a hindered phenol, and batch 3 1 wt % Chimasorb 944, which is a hindered amine. All additives were supplied by Ciba Specialty Chemicals. Batch 4 did not contain any additive. All four batches were extruded before the injection moulding to ensure good mixing. Irganox 1330 and Chimasorb 944 have Formulas II and III.

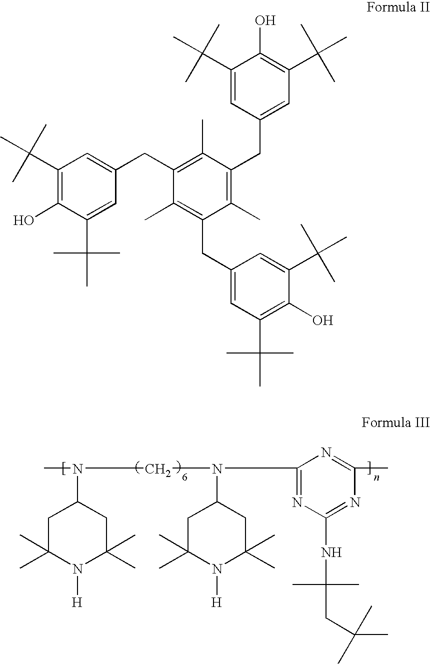

The results from recalculating 1 wt % into the concentration of reactive groups, i.e. the hindered phenol for Irganox 1010 and 1330 and the hindered amine for Chimasorb 944, are shown in Table 1.

| | Concentration of reactive groups |
|---|---|
| Additive | (mmol/g) |
| Irganox 1010 | 61 |
| Irganox 1330 | 69 |
| Chimasorb 944 | 90 |

In addition to the experiments performed on PVDF, 1 wt % Irganox 1010 was also added to commercial grades of ECTFE (Halar 901) supplied by Solvay Solexis.

### Methods of Penetration Depth Measurements

### 1. Indicator Technique

This technique is based on the colour change of a pH-indicator solution. A solution of methyl red in ethanol and acetone was used. Methyl red changes colour from yellow to red when the pH drops below 4.5. It has been found that this technique works very well for determining the penetration depth and diffusion rate of both strong acids and chlorine dioxide.

Samples, approximately 20×20 mm, were cut from the compression moulded plates. The samples were then immersed in the penetrating media and kept at 50° C. The exposure time was between 17 and 24 hours, unless otherwise specified. After exposure the samples were cut in two, and 150 μm thick films were cut from the cross-section with a microtome. The films were then immersed in the methyl red solution. After the reaction time, the samples were cleaned with ethanol and dried. The films were scanned and analysed with image analysis software, giving a colour profile of the penetration depth.

### 2. LGB Method

The LGB-method uses the fact that a buffered solution of Lissamine Green B (LGB) loses its clear blue colour in a reaction with ClO₂. By measuring the absorbance of a ClO₂-containing sample relative to a reference sample and using a calibration curve, the ClO₂ concentration in the initial sample can be calculated.

Slices cut from the sample were put in LGB-solution and then left for 48 hours to ensure that all ClO₂ had diffused out of the samples and reacted with the LGB. After that, the absorbance at 616 nm was measured and the concentration of ClO₂ in the sample slices could be calculated. With these data a concentration profile is obtained by plotting the ClO₂ concentration of the slices versus their cumulative thickness.

### 3. Exposure Technique

When exposed in the industrial environment the samples are mounted on metal bars immersed in the penetrating medium. Thus, the samples are exposed to the medium from both sides and the maximum penetration depth is 1.5 mm into the 3 mm thick samples.

### Results

### Penetration Depth of ClO₂

Samples of PVDF were first immersed in 7 g/l ClO₂ for 17 hours and then microtome slices were cut from the cross-sections and treated with methyl red solution, as described in the experimental part. From the pictures taken it is clear that the penetration depth is reduced substantially by the addition of Irganox 1010 to the sample. It also seems as if the boundary between the ClO₂-affected part and the unaffected core is sharper in the sample with the additive.

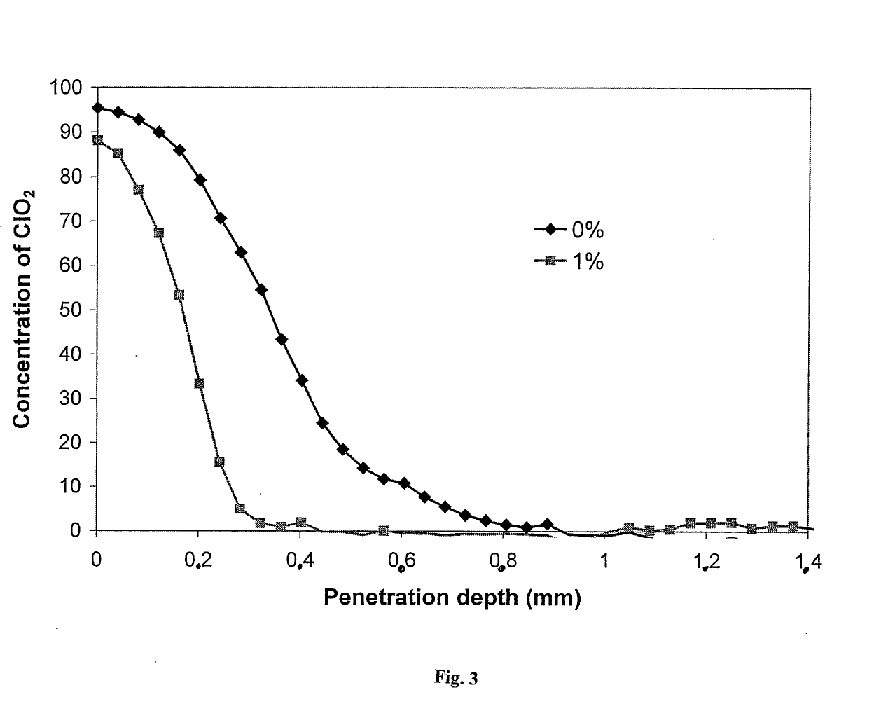

By using the colour analysis program, the concentration of chlorine dioxide can be plotted as a function of penetration depth for the two samples, treated by immersion in 7 g/l ClO₂ solution at 50° C. for 17 hours. One sample contained no additive and the second sample contained 1 wt % Irganox 1010. The results are shown in FIG. 3.

The plots in FIG. 3 show that the profile of the penetration depth is much sharper for the sample with additive than in the one without. In the sample without additive ClO₂ has penetrated 0.8 mm while in the sample with 1 wt % Irganox 1010 the penetration depth is only 0.3 mm. To verify that these results are really valid for the ClO₂ penetration and are not due to some artefact the penetration depth was also followed using the LGB method. This method does not only give a relative concentration of ClO₂ in the sample but can be used to calculate the actual amount in the material.

The results from the indicator technique shown in FIG. 3 and the LGB method give about the same penetration depth of ClO₂.

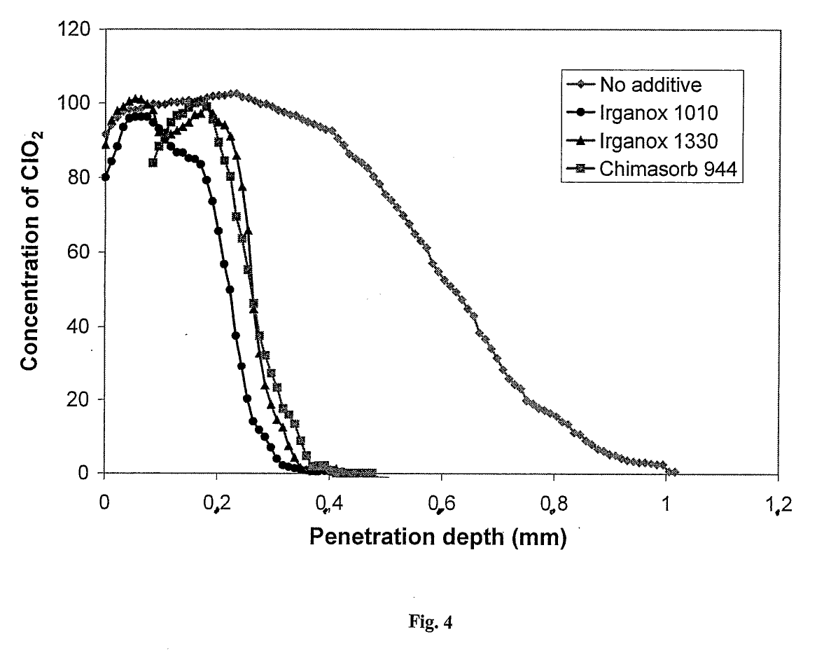

It was found that the addition of both Irganox 1330 and Chimasorb 944 gave about the same effect on the diffusion rate as Irganox 1010 as can be seen in FIG. 4. Observe that the data in FIG. 4 are for a 24 hour exposure at 50° C. while the previously presented data shown in FIG. 3 were from a 17 hour exposure.

When plotting the penetration depth as a function of the concentration of Irganox 1010 it was found to be linear. An extrapolation indicated that an addition of a bit more than 2 wt % Irganox 1010 would result in no penetration at all of ClO₂.

### Consumption of Additive

During the reaction between Irganox 1010 and chlorine dioxide the hindered phenol is destroyed. Normally the detection limit for IR-spectroscopy is quite high and it is difficult to detect low concentrations of additives. However, due to the fact that the hindered phenol group has an IR-absorption in a region where it does not overlap with the PVDF polymer, it was found that it was possible to detect the additive with FTIR. Experiments on ECTFE with 1 wt % Irganox 1010 have shown that it is only the hindered phenol group that decreases and not the ester.

### Exposure in ClO₂ Stripper at Aspa Bruk

Test pieces of PVDF were also exposed 50 days in the ClO₂ stripper at Aspa bruk. The temperature was 58° C. and the ClO₂ concentration about 1.4 g/l. When examining the pieces with indicator solution it was found that the ClO₂ had permeated all the way through all samples except the ones with 0.5 and 1 wt % of additive. In the samples containing the additive no blisters, no other deterioration nor any negative effect on material properties were observed.

Exposure in the Primary ClO₂ Reactor at the Skärblacka Mill

Test pieces of PVDF with different concentrations of Irganox 1010 were also exposed for one year in the primary chlorine dioxide reactor at the Skärblacka mill. The temperature was 58° C. and the ClO₂ concentration around 2.6 g/l.

It was found that the sample without any stabiliser was so brittle that it had fallen apart during the exposure. The addition of Irganox 1010 made the material less susceptible to this attack. Table 2 lists the amount of degraded material in the samples for different concentrations of additive. Above 0.5 wt % no degradation could be found at all. Chlorine dioxide had penetrated all the way through all the samples. For the samples with 0.5 and 1 wt % additive, no blisters, no other deterioration nor any negative effect on material properties were observed after being exposed for one year. It seems that a higher stability was obtained by the additive.

TABLE 2
Amount of degraded material in the samples with different
concentration of additive after exposure for one year
in the primary ClO₂ reactor at the Skärblacka mill.

| Concentration of additive (%) | Amount of degraded material (%) |
|---|---|
| 0 | 100 |
| 0.1 | 100 |
| 0.2 | 40 |
| 0.5 | 0 |
| 1 | 0 |

Exposure in the Pulp/ClO₂ Inlet Line at the Skärblacka Mill

Exposure of test pieces of PVDF with 1 wt % Irganox 1010 and 1330 and Chimasorb 944 and without additive was made in a test station in the by-pass line for pulp/ClO₂ inlet line to the D₀ stage bleach tower at the Skärblacka mill. The temperature is 68-75° C. and the chlorine dioxide charge is 20 kg ClO₂ (as active chlorine) per ton pulp and the pulp consistency is 5%. The samples were exposed for 6 months.

The chlorine dioxide had penetrated all the way through the sample without additive and had only just penetrated into the middle of the one with Chimasorb 944. In the samples with 1 wt % Irganox 1010 or 1330 the diffusion of chlorine dioxide had been slowed down so much that both showed a penetration depth of only 1 mm. This confirms that the addition of the additive is slowing down the diffusion through the fluoroplastic also in an industrial environment and that it does not influence the material in a negative way even after as long exposure times as 6 months. It also shows that the hindered phenols are more active than the hindered amine.

### Exposure in Hot Wet Cl₂-Gas at the Chlorine Plant in Skoghall

Test pieces of PVDF with 1 wt % Irganox 1010 and 1330 and without additive were exposed in hot (75-85° C.) wet chlorine gas at the chlorine plant in Skoghall for 6 months. It was found that the chlorine had permeated all the way through all the samples but it was also seen that the Irganox had been consumed in the samples with the additive. This indicates that the diffusion rate had been decreased for these samples due to the chemical reaction between the chlorine and the hindered phenol. It was also found that the PVDF sample without additive showed an internal layer with cracks due to degraded material. This was not found in the samples with additive and just as for the other industrially exposed samples, no blisters nor any other negative effect on material properties were observed in these samples after the exposure.

### Penetration Depth of HClO

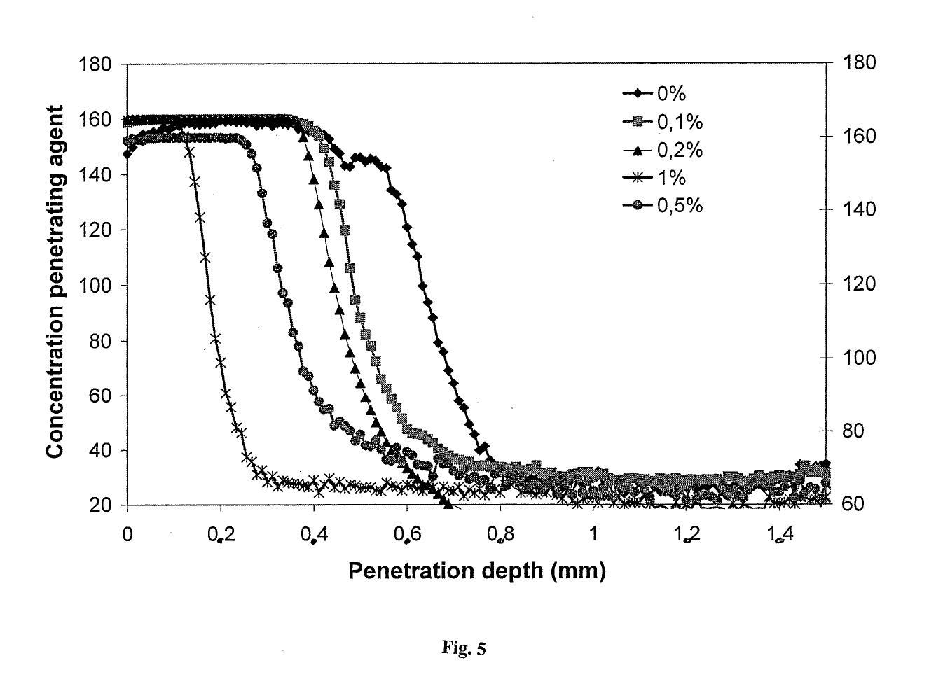

To test if the very promising results of slowing down the diffusion of chlorine dioxide by the addition of Irganox 1010 to PVDF also are applicable to other chlorine species the samples were exposed to a HClO solution at pH 5.5. FIG. 5 shows the profiles from the reaction with the indicator solution after the sample exposure.

As can be seen in the figure the penetration depth is greatly affected by the additive.

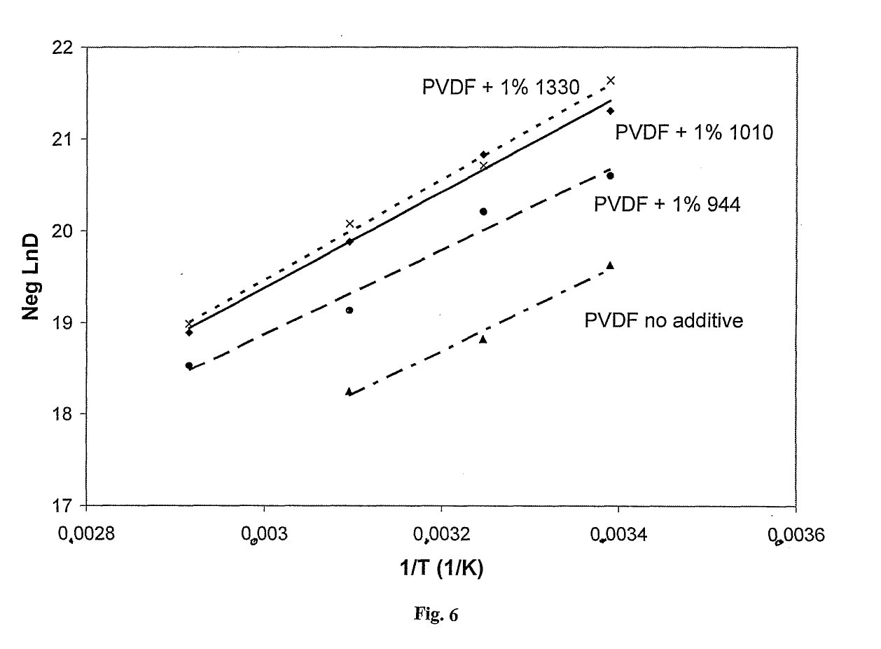

FIG. 6 is a diagram showing Arrhenius plots of the temperature dependence showing the negative logarithm of the diffusion coefficient D as a function of the reciprocal absolute temperature. The higher the value of neg Log D the slower the diffusion. As can be seen in the figure PVDF with Irganox 1330 and Irganox 1010 are, within the experimental error, more or less as efficient throughout the whole temperature range. The hindered amine Chimasorb 944 is a bit less efficient and PVDF without additive shows the highest diffusion rate. It is interesting to note that the slope of the lines is about the same for all mixes, which means that the activation energy is the same. From this it can be concluded that it is the activation energy for the diffusion which is rate determining, i.e. slower than the reaction between the additives and ClO₂.

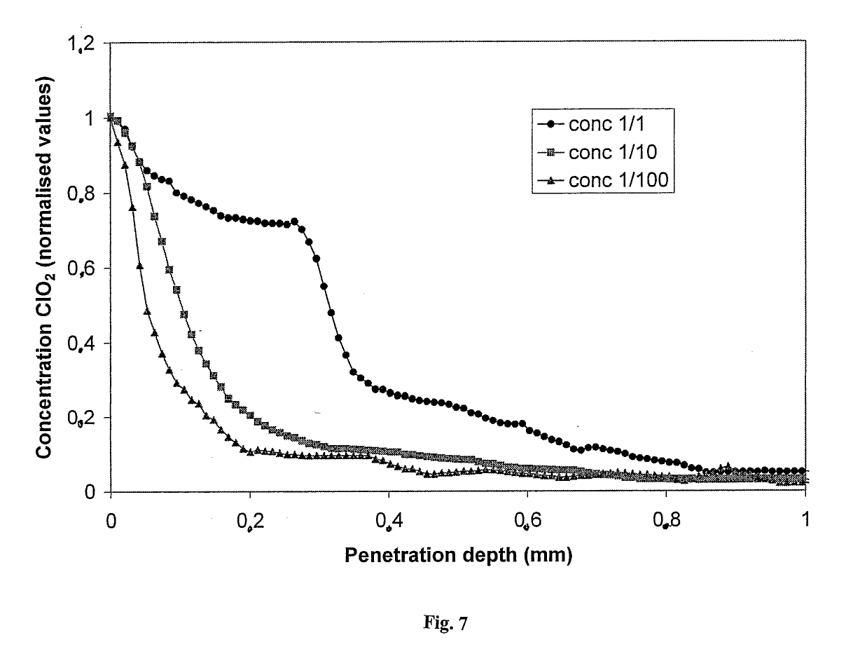

Lastly, FIG. 7 shows the effect of the concentration of the chlorine dioxide solution on the penetration depth in the PVDF containing 1 wt % Irganox 1010. The samples were immersed in the ClO₂-solution at 50° C. for 24 hours. The concentrations used were 3 g/l (conc 1/1), 0.3 g/l (conc 1/10) and 0.03 g/l (conc 1/100), respectively.

### Discussion

It is obvious from the results that an addition of as low as 0.1 wt % Irganox 1010 can slow down the penetration rate of chlorine dioxide and HClO solution. FTIR data show that there is a consumption of the hindered phenol.

It was also found that another hindered phenol, Irganox 1330, gave the same effect as Irganox 1010. This confirms that it is the phenol group that is the active site. Also the hindered amine Chimasorb 944 slows down the diffusion by reacting with the ClO₂ and HClO.

The exposures in the stripper at Aspa bruk and in the pulp/ClO₂ inlet line at the Skärblacka mill show that the addition of reactive additives such as Irganox 1010 does slow down the penetration of ClO₂ also in an industrial environment and for longer ageing times. No blisters or negative effects on material properties were observed.

The exposure in hot wet Cl₂-gas at the chlorine plant in Skoghall shows that the hindered phenols also react with Cl₂ and by this slows down the penetration rate through the material. It was also found that it prevents the material from forming an internal brittle layer.

The reactive additive also stabilised the samples against degradation when exposed in a chlorine dioxide reactor. Addition of at least 0.5 wt % Irganox 1010 completely prevented the degradation of PVDF when exposed for one year.

It seems that the effect of the additive is even higher against HClO than ClOC₂. For HClO it is, however, not clear exactly what the penetrating species is. HClO is a complex mixture of chlorine species and the composition depends on the pH value and the temperature.

### CONCLUSIONS

It is possible to slow down the diffusion of chlorine, chlorine dioxide and HClO in fluoroplastics such as PVDF by adding a reactive additive having reactive groups, such as a hindered phenol.

Further, a stabilization of fluoriplastics such as PVDF and ECTFE in a chlorine dioxide reactor may be obtained by such an additive.

The above discussion and experiments performed mainly on PVDF using some special reactive additives are valid for fluoroplastics in general and in general for any reactive additive with groups which can react with the penetrant.

1. A process of slowing down diffusion of an element or a compound through a fluoroplastic comprising the addition of a reactive additive having reactive groups that react with the element or compound.

2. A process according to claim 1 wherein the element or compound is chlorine or a chlorine compound.

3. A process of preventing degradation of the fluoroplastics polyvinylidene fluoride and ethylene chlorotrifluoroethylene used in, or in connection with a reactor where chlorine dioxide is produced comprising mixing the fluoroplastic with a reactive additive having reactive groups.

4. A process according to claim 1, wherein the reactive additive is a hindered phenol or hindered amine.

5. A process according to claim 4, wherein the reactive additive has one of the formulas:

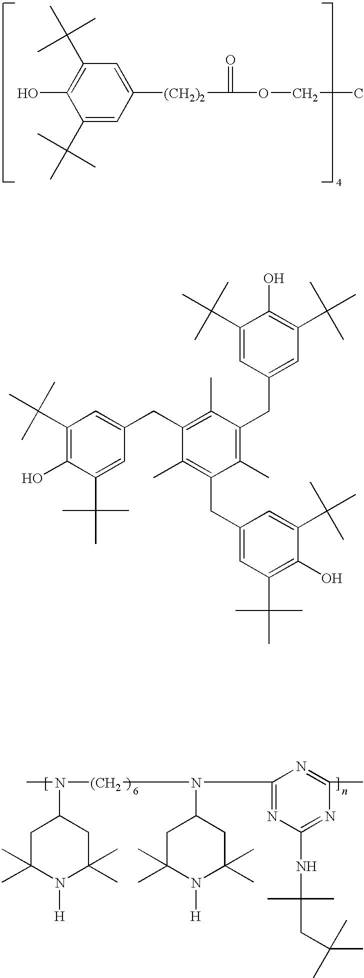

6. A process according to claim 1, wherein the fluoroplastic is to be used as liner on a tank or pipe of steel or fiberglass reinforced plastic.

7. A process according to claim 1, wherein at least 0.1 wt % additive is used.

8. A process according to claim 7, wherein at least 0.5 wt %, additive is used.

9. A process according to claim 1, wherein such an amount of reactive additive is used as to give the fluoroplastic a concentration of reactive groups of at least 6 mmol/g.

10. A diffusion resistant fluoroplastic which comprises a reactive additive having reactive groups that react with an element or compound to which the fluoroplastic is diffusion resistant.

11. A diffusion resistant fluoroplastic according to claim 10, wherein the element or compound is chlorine or a chlorine compound.

12. A diffusion resistant fluoroplastic according to claim 10, wherein the reactive additive is a hindered phenol or hindered amine.

13. A diffusion resistant fluoroplastic according to claim 12, wherein the reactive additive has one of the formulas:

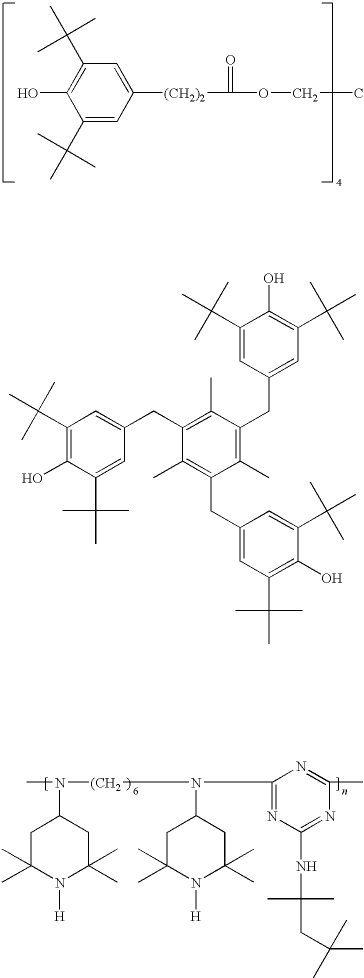

14. A diffusion resistant fluoroplastic according to claim 10, comprising at least 0.1 wt % additive.

15. A diffusion resistant fluoroplastic according to claim 14, comprising at least 0.5 wt % additive.

16. A diffusion resistant fluoroplastic according to claim 10, comprising such an amount of additive as to give the fluoroplastic a concentration of reactive groups of at least 6 mmol/g.

17. A diffusion resistant fluoroplastic according to claim 1 which is essentially free from alkali metal formate, inorganic non-metallic reducing agent, aliphatic polyol, and glycidyl methacrylate polymer.

18. An additive for use in a fluoroplastic to prevent or slow down the diffusion through the fluoroplastic of an element or a compound, the additive comprising a reactive compound having reactive groups, wherein the element or compound reacts with reactive groups.

19. The additive according to claim 18, wherein the element or compound is chlorine or a chlorine compound.

20. An additive for use in the fluoroplastics polyvinylidene fluoride and ethylene chlorotrifluoroethylene to prevent or slow down degradation of the fluoroplastic, the additive comprising a reactive compound having reactive groups, wherein the polyvinylidene fluoride and ethylene chlorotrifluoroethylene are utilized in, or in connection with a reactor for producing chlorine dioxide.

21. The additive according to claim 18, wherein the reactive compound having reactive groups is a hindered phenol or amine.

22. The additive according to claim 18, wherein the fluoroplastic is used as liner on a tank or pipe of steel or fiberglass reinforced plastic.

23. The additive according to claim 18 wherein at least 0.1 wt % additive is used in the fluoroplastic.

24. The additive according to claim 18, wherein such an amount of hindered phenol or amine is used as additive as to give the fluoroplastic a concentration of reactive groups of at least 6 mmol/g.

25. The additive according to claim 18, wherein the hindered phenol or amine has one of the formulas: [chemical structure]
